(19) United States
(12) Patent Application Publication
(10) Pub. No.: US 2006/0088514 A1
(43) Pub. Date: Apr. 27, 2006

(54) Formulation comprising a bacterial strain

(76) Inventors: Liam O'Mahony, Rathcormac (IE); Fergus Shanahan, Kinsale (IE); Barry Kiely, Passage West (IE); John Kevin Collins, Doughcloyne (IE); Gerald Christopher O'Sullivan, Bishopstown (IE)

Correspondence Address: JACOBSON HOLMAN PLLC, 400 SEVENTH STREET N.W., SUITE 600, WASHINGTON, DC, 20004, US

(21) Appl. No.: 11/238,092

(22) Filed: Sep. 29, 2005

Related U.S. Application Data
(63) Continuation of application No. PCT/IE04/00050, filed on Mar. 31, 2004.

(30) Foreign Application Priority Data
Mar. 31, 2003 (IE) 2003/0236

(51) Int. Cl. A61K 35/74

(52) U.S. Cl. 424/93.45

(57) ABSTRACT

An injectable formulation of a bacterial strain such as a Lactobacillus salivarius strain is useful in treating of inflammatory disorders such as colitis or arthritis.

This is a continuation of PCT/IE04/000050 filed Mar. 31, 2004 and published in English.

The invention relates to a formulation comprising a bacterial strain.

### INTRODUCTION

The defence mechanisms to protect the human gastrointestinal tract from colonization by intestinal bacteria are highly complex and involve both immunological and non-immunological aspects (V. J. McCracken and H. R. Gaskins, ‘Probiotics a critical review’, Horizon Scientific Press, UK, 1999, p. 278.). Innate defence mechanisms include the low pH of the stomach, bile salts, peristalsis, mucin layers and anti-microbial compounds such as lysozyme (D. C. Savage, ‘Microbial Ecology of the Gut’, Academic Press, London, 1997, p. 278.). Immunological mechanisms include specialized lymphoid aggregates, underlying M cells, called peyers patches, which are distributed throughout the small intestine and colon (M. F. Kagnoff. Gastroenterol. 1993, 105, 1275). Luminal antigens presented at these sites result in stimulation of appropriate T and B cell subsets with establishment of cytokine networks and secretion of antibodies into the gastrointestinal tract (M. R. Neutra and J-P Kraehenbuhl. ‘Essentials of mucosal immunology’, Academic Press, San Diego, 1996, p. 29., M. E. Lamm. Ann. Rev. Microbiol. 1997, 51, 311). In addition, antigen presentation may occur via epithelial cells to intraepithelial lymphocytes and to the underlying lamina propria immune cells (S. Raychaudhuri et al. Nat Biotechnol., 1998, 16, 1025). Therefore, the host invests substantially in immunological defence of the gastrointestinal tract. However, as the gastrointestinal mucosa is the largest surface at which the host interacts with the external environment, specific control mechanisms must be in place to regulate immune responsiveness to the 100 tons of food, which is handled by the gastrointestinal tract over an average lifetime (F. Shanahan, ‘Physiology of the gastrointestinal tract’, Raven Press, 1994, p. 643.). Furthermore, the gut is colonized by over 500 species of bacteria numbering 10¹¹-10¹²/g in the colon. Thus, these control mechanisms must be capable of distinguishing non-pathogenic adherent bacteria from invasive pathogens, which would cause significant damage to the host. In fact, the intestinal flora contributes to defence of the host by competing with newly ingested potentially pathogenic micro-organisms.

Bacteria present in the human gastrointestinal tract can promote inflammation. Aberrant immune responses to the indigenous microflora have been implicated in certain disease states, such as inflammatory bowel disease (Brandzeag P. et al. Springer Semin. Immunopathol. 1997, 18, 555). Antigens associated with the normal flora usually lead to immunological tolerance and failure to achieve this tolerance is a major mechanism of mucosal inflammation (Stallmach A. et al., Immunuol. Today, 1998, 19, 438). Evidence for this breakdown in tolerance includes an increase in antibody levels directed against the gut flora in patients with IBD.

One of the mechanisms whereby probiotic organisms may protect against mucosal inflammation directly or indirectly is through interaction with the mucosal epithelium and associated lymphoid structures, thereby causing the host to up-regulate and express molecules, which are anti-inflammatory. These would include cytokines such as IL-10 and TGF.

There is a need for formulations for treating inflammatory effects.

The invention is directed towards a formulation of probiotic bacteria, especially to attenuate inflammation.

### STATEMENTS OF INVENTION

According to the invention there is provided an injectable formulation comprising a bacterial strain or an active derivative, fragment or mutant thereof.

In one embodiment the strain is a probiotic bacterial strain.

The strain may be a lactobacillus strain such as a Lactobacillus salivarius strain. One particular strain is Lactobacillus salivarius UCC118.

The strain may be in the form of bacterial cells. The cells may be live/viable cells or dead/non-viable cells.

In one embodiment the formulation comprises a single strain.

In another embodiment the formulation comprises at least two different strains of the same or different species/genus or sub-genus.

The formulation may comprise a prebiotic material.

The invention also provides a vaccine comprising a formulation of the invention.

In another aspect the invention provides an injectable formulation of immunomodulatory bacteria.

The invention further provides use of a formulation of the invention in the prevention and/or treatment of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimier's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system; renal disease, disorders of the central nervous system, hepatic disease, ischaemia, nutritional disorders, osteoporosis, endocrine disorders, epidermal disorders, psoriasis and/or acne vulgaris.

The invention also provides use of a formulation of the invention in the prevention and/or treatment of disorders associated with intestinal inflammation.

In a further embodiment the invention provides use of a formulation of the invention in the prevention and/or treatment of colitis.

In another aspect the invention provides use of a formulation of the invention in the prevention and/or treatment of arthritis.

In a further aspect the invention provides a method for the prophylaxis and/or treatment of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris comprising administering a formulation of the invention.

The invention also provides a method for the prophylaxis and/or treatment of disorders associated with intestinal inflammation comprising administering a formulation of the invention.

The invention further provides a method for the prophylaxis and/or treatment of colitis comprising administering a formulation of the invention.

In another aspect the invention provides a method for the prophylaxis and/or treatment of arthritis comprising administering a formulation of the invention.

The invention provides a method to attenuate inflammation using parenteral administration of bacterial strain(s).

Particular strains of commensal micro-organisms elicit immunomodulatory effects in vitro.

The invention has potential therapeutic value in the prophylaxis or treatment of dysregulated immunological control, such as undesirable inflammatory reactions (e.g. IBS).

Bifidobacteria and lactobacilli are commensal micro-organisms. They have been isolated from the microbial flora within the human gastrointestinal tract. The immune system within the gastrointestinal tract cannot have a pronounced reaction to members of this flora, as the resulting inflammatory activity would also destroy host cells and tissue function. Therefore, some mechanism(s) exist whereby the immune system can recognise commensal non-pathogenic members of the gastrointestinal flora as being different to pathogenic organisms. This ensures that damage to host tissues is restricted and a defensive barrier is still maintained.

The Lactobacillus and Bifidobacterium strains may be a genetically modified mutant or it may be a naturally occurring variant thereof.

Preferably the Lactobacillus and Bifidobacterium strains is in the form of viable cells.

A deposit of Lactobacillus salivarius strain UCC 118 was made at the NCIMB on Nov. 27, 1996 and accorded the accession number NCIMB 40829. The strain of Lactobacillus salivarius is described in WO-A-98/35014.

Other Lactobacillus salivarius strains are described in our WO03/010298A. These include the following:—AH102; AH103; AH105; AH109; AH110.

A deposit of Lactobacillus salivarius strain AH102 was made at the National Collections of Industrial and Marine Bacteria Limited (NCIMB) on Apr. 20, 2000 and accorded the accession number NCIMB 41044.

A deposit of Lactobacillus salivarius strain AH103 was made at the NCIMB on Apr. 20, 2000 and accorded the accession number NCIMB 41045.

A deposit of Lactobacillus salivarius strain AH05 was made at the NCIMB on Apr. 20, 2000 and accorded the accession number NCIMB 41047.

A deposit of Lactobacillus salivarius strain AH109 was made at the NCIMB on Mar. 22, 2001 and accorded the accession number NCIMB 41093.

A deposit of Lactobacillus salivarius strain AH110 was made at the NCIMB on Mar. 22, 2001 and accorded the accession number NCIMB 41094.

The Lactobacillus salivarius may be a genetically modified mutant or it may be a naturally occurring variant thereof.

Preferably the Lactobacillus salivarius is in the form of viable cells. Alternatively the Lactobacillus salivarius may be in the form of non-viable cells.

Other Lactobacillus strains are described in our WO03/010299A. These include:—AH101; AH104; AH111; AH112; AH113.

A deposit of Lactobacillus casei strain AH101 was made at the National Collections of Industrial and Marine Bacteria Limited (NCIMB) on Apr. 20, 2000 and accorded the accession number NCIMB 41043.

A deposit of Lactobacillius casei strain AH104 was made at the NCIMB on Apr. 20, 2000 and accorded the accession number NCIMB; 41046.

A deposit of Lactobacillius casei strain AH111 was made at the NCIMB on Mar. 22, 2001 and accorded the accession number NCIMB 41095.

A deposit of Lactobacillus casei strain AH112 was made at the NCIMB on Mar. 22, 2001 and accorded the accession number NCIMB 41096.

A deposit of Lactobacillus casei strain AH113 was made at the NCIMB on Mar. 22, 2001 and accorded the accession number NCI 41097.

The Lactobacillus casei may be a genetically modified mutant or it may be a naturally occurring variant thereof.

Preferably the Lactobacillus casei is in the form of viable cells. Alternatively the Lactobacillus casei may be in the form of non-viable cells.

A deposit of Bifidobacterium infantis strain 35624 was made at the NCIMB on Jan. 13, 1999 and accorded the accession number NCIMB 41003. The strain of Bifidobacterium infantis is described in WO-A-00/42168.

Other Bifidobacterium strains are described in our WO03/010297A. These include the following:—AH208; AH209; AH210; AH211; AH212 and AH214.

A deposit of Bifidobacterium longum infantis strain AH208 was made at the National Collections of Industrial and Marine Bacteria Limited (NCIMB) on Apr. 20, 2000 and accorded the accession number NCIMB 41050.

A deposit of Bifidobacterium longum infantis strain AH09 was made at the NCIMB on Apr. 20, 2000 and accorded the accession number NCIMB 41051.

A deposit of Bifidobacterium longum infantis strain AH210 was made at the NCIMB on Apr. 20, 2000 and accorded the accession number NCIMB 41052.

A deposit of Bifidobacterium longum infantis strain AH211 was made at the NCIMB on Apr. 20, 2000 and accorded the accession number NCIMB 41053.

A deposit of Bifidobacterium longum infantis strain AH212 was made at the NCIMB on Mar. 22, 2001 and accorded the accession number NCIMB 41099.

A deposit of Bifidobacterium longum infantis strain AH214 was made at the NCIMB on Mar. 22, 2001 and accorded the accession number NCIMB 41100.

The Bifidobacterium longum infantis may be a genetically modified mutant or it may be a naturally occurring variant thereof.

### BRIEF DESCRIPTION OF THE DRAWINGS

FIG. 1 compares the anti-inflammatory efficacy of a probiotic lactobacillus strain following enteral or parenteral administration.

FIG. 2 demonstrates that injection of a lactobacillus strain is superior to oral lactobacillus treatment in a rheumatoid arthritis murine model.

### DETAILED DESCRIPTION

The invention is based on the finding that probiotic strains exert anti-inflammatory effects following administration to a non-mucosal site.

The microflora on mucosal surfaces are vast in number and complexity. Many hundreds of bacterial strains exist and account for approximately 90% of the cells found in the human body, the remainder of the cells being human. The vast majority of these bacterial strains do not cause disease and may actually provide the host with significant health benefits (e.g. Bifidobacteria and lactobacilli). These bacterial strains are termed commensal organisms. Mechanism(s) exist whereby the immune system at mucosal surfaces can recognise commensal non-pathogenic flora as being different to pathogenic organisms.

The human immune system plays a significant role in the aetiology and pathology of a vast range of human diseases. Hyper and hypo-immune responsiveness results in, or is a component of, the majority of disease states. One family of biological entities, termed cytokines, are particularly important to the control of immune processes. Pertubances of these delicate cytokine networks are being increasingly associated with many diseases. These diseases include but are not limited to inflammatory disorders, immunodeficiency, inflammatory bowel disease, irritable bowel syndrome, cancer (particularly those of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and acne vulgaris.

This invention describes the potential of certain bacterial strains in customising host cell cytokine production. In this way customisation of disease specific therapies may be accomplished using a selection of bacterial strains.

Recognition of bacterial species by host cells results in distinct patterns of cytokine production and immune responses. The cytokines produced by host cells are secreted into the extracellular milieu. These cytokines deliver an informative signal to neighbouring cells, which do not necessarily have to be in physical contact with the bacterium. This “bystander” effect results in many different cell types being influenced by the cytokine network established by bacterial stimulated mucosal cells.

As the majority of cytokines may have both pro and anti-inflammatory activities, patterns or networks of cytokine release have been associated with different types of immune responses. The existence of T cells, which differ in their pattern of cytokine secretion, allows differentiation of inflammatory or immune responses into at least three categories, cell mediated or humoral responses or Th3/Tr1 regulatory responses. Th1 responses are categorised by IFN, TNF and IL-2 production leading to a cell-mediated response while Th2 cells secrete IL-4, IL-5, IL-9, IL-10 and IL-13 resulting in a humoral response. Th3/Tr1 responses are characterised by T cell secretion of the regulatory cytokines IL-10 and TGF. Differentiation of T cells into either network depends on the cytokine milieu in which the original antigen priming occurs (Seder et al., 1992). In addition, the polarisation of T cell subpopulations are influenced by a number of other cell types including dendritic cells and epithelial cells. (Mosmann & Sad, 1996). Certain types of stimulation may also direct this response, such as immune complex deposition within inflammatory sites which increases IL-6 and IL-10 production and inhibits production of TNF and IL-1 thus influencing the Th1/Th2 balance. For successful elimination of pathogens, the correct cytokine network needs to be established. For example, the intracellular bacterium Listeria monocytogenes elicits a Th1 response while the extracellular parasite Nippostrongylus brasiliensis requires a Th2 response. Each of these T cell subsets produce cytokines that are autocrine growth factors for that subset and promote differentiation of naive T cells into that subset (Trinchieri et al. 1996). These two subsets also produce cytokines that cross-regulate each other's development and activity. IFN amplifies Th1 development and inhibits proliferation of Th2 T cells while IL-10 blocks Th1 activation. Tr1 cells have a profound suppressive effect on antigen-specific T cell responses mediated by secretion of IL-10 and TGF (Groux et al., 1997) and cytokine independent mechanisms such as direct cell-cell contact.

The cytokine networks involved in immune responses are subject to a complex number of control pathways that normally result in restriction of cellular damage and eradication of the infectious organism. However, unregulated release of these cytokines can have damaging consequences. Incorrect Th1/Th2 responses contribute to the pathogenesis of certain diseases. For instance, the healing form of leprosy (tuberculoid lesion) is associated with a Th1 response while uncontrolled leprosy (lepromatous lesion) is associated with a Th2 response. Chronic inflammatory responses can lead to the death of the host. For instance, rats infected with the protozoan parasite Trypanosoma brucei become cachectic, develop anaemia and eventually die. Production of proinflammatory cytokines has been associated with the pathogenesis of many disorders. In Langerhans cell histiocytosis, cytokines may be involved in some of the tissue damage seen with this disease (Kannourakis & Abbas, 1994). Rheumatoid arthritis is a chronic inflammatory disease of the synovial joints resulting in cartilage destruction and bone erosion (Kouskoff et al., 1996). Elevated levels of proinflammatory cytokines have been detected from patients with rheumatoid arthritis and these levels could be associated with disease activity, altered energy metabolism and food intake (Roubenoff et al., 1994). In patients with sepsis, cardiovascular shock and organ dysfunction may be initiated by the production of proinflammatory cytokines stimulated by the infectious organism particularly in patients with cerebral malaria (Kwiatkowski et al., 1990). Certain alleles of polymorphic sites associated with TNF production have been shown to predict patients with cerebral malaria (McGuire et al., 1994) and severe sepsis (Stuber et al., 1996) who will be most adversely affected. Genetic predisposition to increased TNF production may also be associated with the development of autoimmune diseases such as diabetes and systemic lupus erythematosus. Inhibition of proinflammatory cytokine production has reduced the damage caused by many disease states. IL-1RA reduces the severity of diseases such as shock, lethal sepsis, inflammatory bowel disease, experimental arthritis and proliferation of human leukaemic cells (for review see Dinarello, 1992). Inhibition of TNF in septic shock prevents the syndrome of shock and tissue injury despite persistent bacteraemia in animal models. Loss of the TNF receptor type I in knock-out mice protects against endotoxic shock (Pfeiffer et al., 1993). Anti-cytokine strategies in humans with sepsis have yielded disappointing results possibly due to complications such as the late administration of these factors after the initial inflammatory insult. However, studies involving neutralising TNF antibodies in rheumatoid arthritis and Crohn's disease have had considerable success with significant reductions in disease activity being observed (Moreland et al., 1997, Stack et al., 1997). Inhibition of transcription factors, such as NF-B, which are responsible for intracellular signalling in the inflammatory response have been successful in reducing tissue damage in animals with chronic intestinal inflammation (Neurath et al., 1996). Moreover, adoptive transfer of T cells secreting IL-10 inhibited colitis in a murine model (Asserman et al., 1999). In addition, consumption of certain bacterial strains results in attenuation of gastrointestinal inflammatory activity (O'Mahony et al., 2001, Rembacken et al., 1999). Therefore, while the inflammatory response is critical to the defence and repair of host tissues, uncontrolled responses can result in significant tissue and organ damage and may result in the death of the host.

TGF refers to a family of closely related molecules termed TGF 1 to −5 (Roberts & Sporn, 1990). All are released from cells in a biologically inactive form due to their association with a latency protein which is believed to be a critical regulatory step. Three receptors have been identified for TGF. Only two of these receptors transduce an intracellular signal suggesting a decoy function for the third receptor. Like the MIP family, TGF also functions as a chemotactic factor for both monocytes and neutrophils. However, this cytokine has diverse effects as both pro and anti-inflammatory effects have been described. Aggregated platelets following vascular injury release TGF resulting in inflammatory cell recruitment to the tissue. Activated monocytes and neutrophils synthesize TGF further increasing cellular recruitment. Monocyte integrin expression is also enhanced by TGF as is the induction of collagenase type IV which may aid movement through basement membranes into inflamed sites (Wahl et al., 1993). TGF increases the expression of Fc RIII (CD16) which recognises antibody bound cells thereby increasing phagocytic activity. The production of inflammatory cytokines by monocytes can also be stimulated by TGF. However, expression of IL-1 receptor antagonist (IL-1RA) is also increased suggesting that this cascade, in part, may be self regulating. TGF is also important as a negative regulatory agent. It antagonises the effects of many inflammatory cytokines and inhibits the proliferation of thymocytes, B cells and haemapoietic stem cells. The activity of a number of cell types can be suppressed by TGF including natural killer (NK) cells, cytotoxic T lymphocytes and lymphokine activated killer (LAK) cells. TGF also has suppressive effects on the release of reactive oxygen and nitrogen intermediates by tissue macrophages (Ding et al., 1990). The immune inhibitory effects of TGF can most clearly be observed in its effects on diseases such as experimental arthritis, multiple sclerosis and graft rejection. Through the stimulation of matrix protein production, TGF may be important to wound healing which is also indicated by its chemotactic activity for fibroblasts (Roberts & Sporn, 1990). Therefore TGF may have important functions with regard to resolution of the inflammatory response and promotion of healing within the inflammatory lesion.

IL-10 is produced by T cells, B cells, monocytes and macrophages (De Waal Malefyt et al., 1991). This cytokine augments the proliferation and differentiation of B cells into antibody secreting cells (Go et al., 1990). IL-10 exhibits mostly anti-inflammatory activities. It up-regulates IL-1RA expression by monocytes and suppresses the majority of monocyte inflammatory activities. IL-10 inhibits monocyte production of cytokines, reactive oxygen and nitrogen intermediates, MHC class II expression, parasite killing and IL-10 production via a feed back mechanism (De Waal Malefyt et al., 1991). This cytokine has also been shown to block monocyte production of intestinal collagenase and type IV collagenase by interfering with a PGE₂-cAMP dependant pathway (Mertz et al., 1994) and therefore may be an important regulator of the connective tissue destruction seen in chronic inflammatory diseases.

TNF is a proinflammatory cytokine, which mediates many of the local and systemic effects seen during an inflammatory response. This cytokine is primarily a monocyte or macrophage derived product but other cell types including lymphocytes, neutrophils, NK cells, mast cells, astrocytes, epithelial cells (Neale et al., 1995) endothelial cells and smooth muscle cells can also synthesise TNF. TNF is synthesised as a prohormone and following processing the mature 17.5 kDa species can be observed. Purified TNF has been observed as dimers, trimers and pentamers with the trimeric form postulated to be the active form in vivo. Three receptors have been identified for TNF. A soluble receptor seems to function as a TNF inhibitor while two membrane bound forms have been identified with molecular sizes of 60 and 80 kDa respectively (Schall et al., 1990). Local TNF production at inflammatory sites can be induced with endotoxin and the glucocorticoid dexamethasone inhibits cytokine production. TNF production results in the stimulation of many cell types. Significant anti-viral effects could be observed in TNF treated cell lines and the IFNs synergise with TNF enhancing this effect (Wong & Goeddel, 1986). Endothelial cells stimulated by TNF produce procoagulant activity, expression of adhesion molecules, IL-1, hematopoitic growth factors, platelet activating factor (PAF) and arachidonic acid metabolites. TNF stimulates neutrophil adherence, phagocytosis, degranulation, reactive oxygen intermediate production and may influence cellular migration (Livingston et al., 1989). Leucocyte synthesis of GM-CSF, TGF, IL-1, IL-6, PGE₂ and TNF itself can all be stimulated upon TNF administration (Cicco et al., 1990). Programmed cell death (apoptosis) can be delayed in monocytes. (Mangan et al., 1991) while effects on fibroblasts include the promotion of chemotaxis and L-6, PGE₂ and collagenase synthesis. While local TNF production promotes wound healing and immune responses, the dis-regulated systemic release of TNF can be severely toxic with effects such as cachexia, fever and acute phase protein production being observed (Dinarello et al., 1988).

Interferon-gamma (IFN is primarily a product of activated T lymphocytes and due to variable glycosylation it can be found ranging from 20 to 25 kDa in size. This cytokine synergizes with other cytokines resulting in a more potent stimulation of monocytes, macrophages, neutrophils and endothelial cells. IFN also amplifies lipopolysaccharide (LPS) induction of monocytes and macrophages by increasing cytokine production, increased reactive intermediate release, phagocytosis and cytotoxicity. IFN induces, or enhances the expression of major histocompatibility complex class II (MHC class II) antigens on monocytic cells and cells of epithelial, endothelial and connective tissue origin (Arai et al., 1990). This allows for greater presentation of antigen to the immune system from cells within inflamed tissues. IFN may also have anti-inflammatory effects. This cytokine inhibits phospholipase A₂, thereby decreasing monocyte production of PGE₂ and collagenase (Wahl et al., 1990). IFN may also modulate monocyte and macrophage receptor expression for TGF, TNF and C5a thereby contributing to the anti-inflammatory nature of this cytokine. Probiotic stimulation of this cytokine would have variable effects in vivo depending on the current inflammatory state of the host, stimulation of other cytokines and the route of administration.

IL-12 is a heterodimeric protein of 70 d) composed of two covalently linked chains of 35 kD and 40 kD. It is produced primarily by antigen presenting cells, such as macrophages, early in the inflammatory cascade. Intracellular bacteria stimulate the production of high levels of IL-12. It is a potent inducer of IFNγ production and activator of natural killer cells. IL-12 is one of the key cytokines necessary for the generation of cell mediated, or Th1, immune responses primarily through its ability to prime cells for high IFN production. IL-12 induces the production of IL-10 which feedback inhibits IL-12 production thus restricting uncontrolled cytokine production. TGF-β also down-regulates IL-12 production. IL-4 and IL-13 can have stimulatory or inhibitory effects on IL-12 production. Inhibition of IL-12 in video may have some therapeutic value in the treatment of Th1 associated inflammatory disorders, such as multiple sclerosis.

The invention will be more clearly understood from the following examples.

### EXAMPLE 1

### Probiotic Attenuation of Colitis in Animal Models

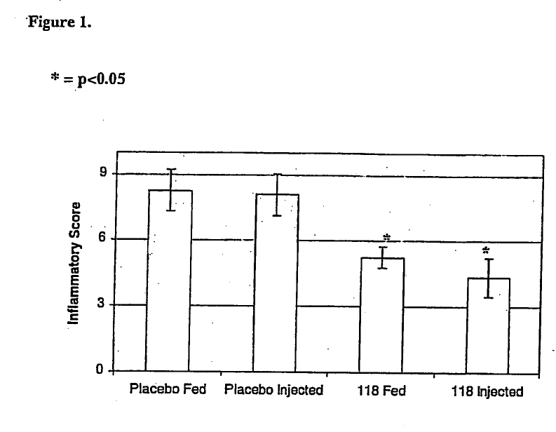

Eighty IL-10 knockout mice were randomised to one of four groups, with 20 mice per group. L. salivarius 118 was administered subcutaneously to one study group, while sterile phosphate buffered saline (PBS) was administered subcutaneously to the control group. L. salivarius 118 was initially grown to a 10 ml volume in de Man, Rogosa, Sharpe (MRS) broth (Oxoid, UK) by incubating overnight at 37° C. under anaerobic conditions. The bacteria was washed twice and resuspended in sterile PBS to a final concentration of 1×10⁹ per ml. A dose of 1×10⁸ bacteria, per mouse, was then injected subcutaneously. These inoculations were performed at weeks 2, 4, 6, 10, 14 and 18 and the mice were sacrificed after 19 weeks. In addition, twenty IL-10KO mice were administered 1×10⁸ L. salivarius 118, or placebo product, daily for 19 weeks. Feeding with both probiotic strains significantly reduced gastrointestinal inflammatory activity (FIG. 1).

An additional group of controls (n=20) receiving heat-treated L. salivarius 118. However, these mice had an obvious and rapid response to the administration of the killed bacteria, becoming shocked within hours. This effect may be related to the method used to kill the bacteria

### EXAMPLE 2

### Probiotic Attenuation of Rheumatoid Arthritis in a Murine Model

Forty-six DBA/1 mice were randomised to five groups. The mice in the 15′ group were injected subcutaneously with L. salivarius UCC118, at a dose of 1×10⁸ bacteria per mouse (preparation as for the IL-10 KO trial.) The 2nd group received an equal volume of sterile PBS administered subcutaneously. Inoculations were administered at weeks 1, 4 and 8. The next two groups were administered 1×10⁸ L. salivarius 118, or placebo, daily. A final group (n=6) did not receive any of the above treatments.

At week 6, arthritis was induced as follows: bovine type II collagen (Chondrex) was dissolved in 0.05M acetic acid to a concentration of 2 mg/ml by stirring overnight at 4° C. This was then emulsified in equal volumes of Freund's complete adjuvant (2 mg/ml of M. tuberculosis strain H37Ra (CFA; Difco)). Mice were immunised subcutaneously, in the tail with 100 μl at week 6. At week 9, a booster immunisation of 50 μl of collagen emulsified in Freund's incomplete adjuvant (IFA; Difco) was administered to all disease groups. From week 10 onwards, mice were assessed on a daily basis for the visual appearance of arthritis in the peripheral joints (18,19). Visual signs were assessed using the following index: 0, normal; 1, mild but definite redness and swelling of the ankle or wrist, or apparent redness and swelling limited to individual digits, regardless of the number of affected digits; 2, moderate redness or swelling of the ankle or wrist; 3,severe redness and swelling of the entire paw including digits; 4, maximally inflamed limb with involvement of multiple joints.

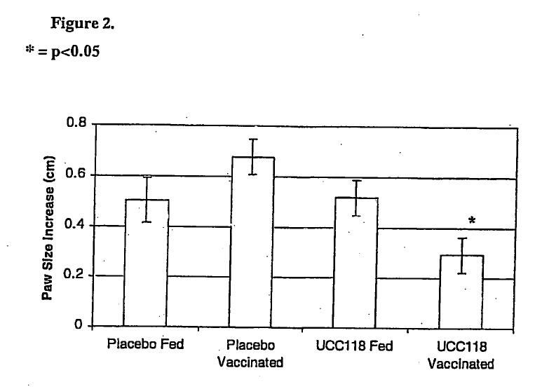

The trial was completed after 12 weeks, at which time all mice were sacrificed by cervical dislocation. At sacrifice, the thickness of each paw was measured using a spring-loaded calliper and joint destruction was measured by histology. Only the animals injected with L. salivarius significantly reduced disease symptoms (FIG. 2). Thus, parenteral treatment with this probiotic strain was superior to enteral administration for treatment of a systemic inflammatory response.

The results confirm that the systemic administration of L. Salivarius 118 had an anti-inflammatory effect on colitis in IL-10 knockout mice. The results are not specific to this model, nor to intestinal inflammation, because the anti-inflammatory effect was also seen in a murine model of arthritis.

Immunomodulation

The human immune system plays a significant role in the aetiology and pathology of a vast range of human diseases. Hyper and hypo-immune responsiveness results in, or is a component of, the majority of disease states. One family of biological entities, termed cytokines, are particularly important to the control of immune processes. Pertubances of these delicate cytokine networks are being increasingly associated with many diseases. These diseases include but are not limited to inflammatory disorders, immunodeficiency, inflammatory bowel disease, irritable bowel syndrome, cancer (particularly those of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and acne vulgaris. The effects on cytokine production are specific for each of the probiotic strains examined. Thus specific probiotic strains may be selected for normalising an exclusive cytokine imbalance particular for a specific disease type. Customisation of disease specific therapies can be accomplished using a selection of the probiotic strains listed above.

Immune Education

The enteric flora is important to the development and proper function of the intestinal immune system. In the absence of an enteric flora, the intestinal immune system is underdeveloped, as demonstrated in germ free animal models, and certain functional parameters are diminished, such as macrophage phagocytic ability and immunoglobulin production (Crabbe et al., 1968, Wostmnann et al., 1996). The importance of the gut flora in stimulating non-damaging immune responses is becoming more evident. The increase in incidence and severity of allergies in the western world has been linked with an increase in hygiene and sanitation, concomitant with a decrease in the number and range of infectious challenges encountered by the host. This lack of immune stimulation may allow the host to react to non-pathogenic, but antigenic, agents resulting in allergy or autoimmunity. Deliberate consumption of a series of non-pathogenic immunomodulatory bacteria would provide the host with the necessary and appropriate educational stimuli for proper development and control of immune function.

Inflammation

Inflammation is the term used to describe the local accumulation of fluid, plasma proteins and white blood cells at a site that has sustained physical damage, infection or where there is an ongoing immune response. Control of the inflammatory response is exerted on a number of levels (for review see Henderson B., and Wilson M. 1998. In “Bacteria-Cytokine interactions in health and disease. Portland Press, 79-130). The controlling factors include cytokines, hormones (e.g. hydrocortisone), prostaglandins, reactive intermediates and leukcotrienes. Cytokines are low molecular weight biologically active proteins that are involved in the generation and control of immunological and inflammatory responses, while also regulating development, tissue repair and haematopoiesis. They provide a means of communication between leukocytes themselves and also with other cell types. Most cytokines are pleiotrophic and express multiple biologically overlapping activities. Cytokine cascades and networks control the inflammatory response rather than the action of a particular cytokine on a particular cell type (Arai K I, et al., Annu Rev Biochem 1990;59:783-836). Waning of the inflammatory response results in lower concentrations of the appropriate activating signals and other inflammatory mediators leading to the cessation of the inflammatory response. TNF is a pivotal proinflammatory cytokine as it initiates a cascade of cytokines and biological effects resulting in the inflammatory state. Therefore, agents which inhibit TNF are currently being used for the treatment of inflammatory diseases, e.g. infliximab.

Pro-inflammatory cytokines are thought to play a major role in the pathogenesis of many inflammatory diseases, including inflammatory bowel disease (IMD). Current therapies for treating IBD are aimed at reducing the levels of these proinflammatory cytokines, including IL-8 and TNFα Such therapies may also play a significant role in the treatment of systemic inflammatory diseases such as rheumatoid arthritis.

The strains of the present invention may have potential application in the treatment of a range of inflammatory diseases, particularly if used in combination with other anti-inflammatory therapies, such as non-steroid anti-inflammatory drugs (NSAIDs) or Infliximab.

This invention describes a novel method for administration of immunomodulatory bacterial. However, this technology can be applied to a number of bacterial types and should not be limited to these bacterial strains alone.

It is unknown whether the complete bacterial cell is required to exert an immunomodulatory effect or if individual active components of the bacterial strains can be utilised alone. Proinflammatory components of certain bacterial strains have been identified. The proinflammatory effects of gram-negative bacteria are mediated by a number of cellular structures including lipopolysaccharide (LPS). LPS alone induces a proinflammatory network. It is assumed that components of probiotic bacteria possess anti-inflammatory activity, due to the effects of the whole cells. Upon isolation of these components, pharmaceutical grade manipulation is anticipated. Therefore the term bacterial strain as used in this specification refers to active components thereof.

The general use of the bacterial strains is in the form of viable cells. However, it can also be extended to non-viable cells such as killed cultures or compositions containing beneficial factors expressed by the bacterial strains. This could include micro-organisms killed by exposure to altered pH or subjection to pressure. With non-viable cells product preparation is simpler, cells may be incorporated easily into pharmaceuticals and storage requirements are not as limited. Lactobacillus casei YIT 9018 offers an example of the effective use of heat killed cells as a method for the treatment and/or prevention of tumour growth as described in U.S. Pat. No. 4,347,240. However, the anti-inflammatory properties disclosed in this application are not retained following heat treatment of these cells.

Other Active Ingredients

It will be appreciated that the probiotic strains may be administered prophylactically or as a method of treatment either on its own or with other probiotic and/or prebiotic materials as described above. In addition, the bacteria may be used as part of a prophylactic or treatment regime using other active materials such as those used for treating inflammation or other disorders especially those with an immunological involvement. Such combinations may be administered in a single formulation or as separate formulations administered at the same or different times and using the same or different routes of administration.

The invention is not limited to the embodiments herein before described which may be varied in detail.

### REFERENCES

Wilmes-Riesenberg M. R., Bearson B., Foster J. W. & Curtiss R. Role of the acid tolerance response in virulence of Salmonella typhimurium. Infect Immun., 1996:1085-92.
Seder R A, Paul W E, Davis M M, Fazekas de St Groth B. The presence of interleukin 4 during in vitro priming determines the lymphokine-producing potential of CD4+ T cells from T cell receptor transgenic mice. J Exp Med 1992 Oct. 1;176(4):1091-8.
Mosmann T. R. & Sad S. The expanding universe of T-cell subsets: Th1, Th2 and more. Immunol. Today, 1996; 17:138-46.
Trinchieri G, Peritt D, Gerosa F. Acute induction and priming for cytokine production in lymphocytes. Cytokine Growth Factor Rev 1996 August;7(2):123-32.
Maldonado-Lopez R., De Smedt T., Michel P., Godfroid J., Pajak B., Heirman C., Thielemans K, Leo O., Urbain J. & Moser M. CD8+ and CD8−subclasses of dendritic cells direct the development of distinct T helper cells in vivo. J. Exp. Med., 1999; 189:587-92.
Groux H, O'Garra A., Bigler M., Rouleau M., Antonenko S., de Vries J. E. & Roncarolo M. G. A CD4+ T-cell subset inhibits antigen-specific T-cell responses and prevents colitis. Nature, 1997; 389:737-42.
Jonuleit H., Schmitt E., Schuler G., Knop J. & Enk A. H. Induction of interleukin 10-producing, nonproliferating CD4+ T cells with regulatory properties by repetitive stimulation with allogeneic immature human dendritic cells. J. Exp. Med., 2000; 192:1213-22.
Kannourakis G. Abbas A. The role of cytokines in the pathogenesis of Langerhans cell histiocytosis. Br J Cancer Suppl 1994 September;23:S37-40.
Kouskoff V, Korganow A S, Duchatelle V, Degott C, Benoist C, Mathis D. Organ-specific disease provoked by systemic autoimmunity. Cell 1996 Nov. 29;87(5):811-22.
Roubenoff R, Roubenoff R A, Cannon J G, Kehayias J J, Zhuang H, Dawson-Hughes B, Dinarello C A, Rosenberg I H. Rheumatoid cachexia: cytokine-driven hypermetabolism accompanying reduced body cell mass in chronic inflammation. J Clin Invest 1994 June;93(6):2379-86.
Kwiatkowski D, Hill A V, Sambou I, Twumasi P, Castracane J, Manogue K R, Cerami A, Brewster D R, Greenwood B M. TNF concentration in fatal cerebral, non-fatal cerebral, and uncomplicated Plasmodium falciparum malaria. Lancet 1990 November 17;336(3725):1201-4.
McGuire W, Hill A V, Allsopp C E, Greenwood B M, Kwiatkowski D. Variation in the TNF-alpha promoter region associated with susceptibility to cerebral malaria Nature 1994 Oct. 6;371(6497):508-10.
Stuber F, Petersen M, Bokelmann P, Schade U. A genomic polymorphism within the tumor necrosis factor locus influences plasma tumor necrosis factor-alpha concentrations and outcome of patients with severe sepsis. Crit Care Med 1996 March;24(3):381-4.
Dinarello C A. The role of interleukin-1 in host responses to infectious diseases. Infect Agents Dis 1992 October;1(5):227-36.
Pfeffer K, Matsuyama T, Kundig T M, Wakeham A, Kishihara K, Shahinian A, Wiegmann K, Ohashi P S, Kronke M, Mak T W. Mice deficient for the 55 kd tumor necrosis factor receptor are resistant to endotoxic shock, yet succumb to L. monocytogenes infection. Cell 1993 May 7;73(3):457-67.
Moreland L W, Baumgartner S W, Schiff N M, Tindall R A, Fleischmann R M, Weaver A L, Ettlinger R E, Cohen S, Koopman W J, Mohler R, Widmer M B, Blosch C M. Treatment of rheumatoid arthritis with a recombinant human tumor necrosis factor receptor (p75)-Fc fusion protein. N Engl J Med 1997 Jul. 17;337(3):141-7.
Stack W A, Mann S D, Roy A J, Heath P, Sopwith M, Freeman J, Holmes G, Long R. Forbes A, Kamm M A. Randomised controlled trial of CDP571 antibody to tumour necrosis factor-alpha in Crohn's disease. Lancet 1997 February 22;349(9051):521-4.
Neurath M F, Pettersson S, Meyer zum Buschenfelde K H, Strober W. Local administration of antisense phosphorothioate oligonucleotides to the p65 subunit of NF-kappa B abrogates established experimental colitis in mice. Nat Med 1996 September;2(9):998-1004.

Asserman C., Mauze S., Leach M. W., Coffman R. L. & Powrie F. An essential role for interleukin 10 in the function of regulatory T cells that inhibit intestinal inflammation. J. Exp. Med., 1999; 190:995-1003.

O'Mahony L, Feeney M, O'Halloran S, Murphy L, Kiely B, Fitzgibbon J, Lee G, O'Sullivan G, Shanahan F, Collins J K. Probiotic impact on microbial flora, inflammation and tumour development in IL-10 knockout mice. Alim. Pharmacol. Ther. 2001; 15(8):1219-1225.
Rembacken B J, Snelling A M, Hawkey P M, Chalmers D M, Axon A T. Non-pathogenic Escherchia coli versus mesalazine for the treatment of ulcerative colitis: a randomised trial. Lancet 1999; 354: 635-9.
Bienenstock J. Mast cell-nerve interactions: possible significance of nerve growth factor. In Mast Cells and Basophils in Physiology, Pathology and Host Defense, ed. Marone G. Lichtenstein L M, Galli S J, Academic Press, 2000.
Matsuda H. Koyama H, Sato H, et al. Role of nerve growth factor in cutaneous wound healing: accelerating effects in normal and healing-impaired diabetic mice. J Exp Med 1998; 187:297-306.
Reinshagen M, Rohm K Steinicamp M, et al. Protective role of neurotrophins in experimental inflammation of the rat gut. Gastroenterology 2000; 119:368-376.
Flugel A, Matsumuro K, Neumann H. et al. Anti-inflammatory activity of nerve growth factor in experimental autoimmune encephalomyelitis: inhibition of monocyte transendothelial migration. Eur J Immunol 2001; 31:11-22.
Roberts A B, Flanders K C, Heine U I, Jakowlew S. Kondaiah P. Kim S J, Sporn M B. Transforming growth factor-beta: multifunctional regulator of differentiation and development. Philos Trans R Soc Lond B Biol Sci 1990 Mar. 12;327(1239):145-54.
Wahl S M, Allen J B, Weeks B S, Wong H L, Klotman P E. Transforming growth factor beta enhances integrin expression and type IV collagenase secretion in human monocytes. Proc Natl Acad Sci USA 1993 May 15;90(10):4577-81.
Ding A, Nathian C F, Graycar J, Derynck R, Stuehr D J, Srimal S. Macrophage deactivating factor and transforming growth factors-beta 1-beta 2 and -beta 3 inhibit induction of macrophage nitrogen oxide synthesis by I-gamma. J Immunol 1990 Aug. 1;145(3):940-4.
Arai K I, Lee F, Miyajima A, Miyatalce S. Arai N. Yokota T. Cytokines: coordinators of immune and inflammatory responses. Annu Rev Biochem 1990;59:783-836.
Aielo F B, Longo D L, Overton R, Takacs L, Durum S K. A role for cytokines in antigen presentation: IL-1 and IL-4 induce accessory functions of antigen-presenting cells. J Immunol 1990 Apr. 1;144(7):2572-81.
Corcoran M L, Stetler-Stevenson W G, Brown P D, Wahl L M. Interleukin 4 inhibition of prostaglandin E2 synthesis blocks interstitial collagenase and 92-kDa type IV collagenase/gelatinase production by human monocytes. J Biol Chem 1992 January. 5;267(1):515-9.
Mangan D F, Welch G R, Wal S M. Lipopolysaccharide, tumor necrosis factor-alpha, and IL-1 beta prevent programmed cell death (apoptosis) in human peripheral blood monocytes. J Immunol 1991 Mar. 1;146(5):1541-6.
Hart P H, Ahern M J, Smith M D, Finlay-Jones J J. Regulatory effects of IL-13 on synovial fluid macrophages and blood monocytes from patients with inflammatory arthritis. Clin Exp Immunol 1995 March,99(3):331-7.
de Waal Malefyt R, Haanen 3, Spits I, Roncarolo M G, te Velde A, Figdor C, Johnson K, Kastelein R. Yssel H. de Vries J E. Interleukin 10 (IL-10) and viral IL-10 strongly reduce antigen-specific human T cell proliferation by diminishing the antigen-presenting capacity of monocytes via downregulation of class II major histocompatibility complex expression. J Exp Med 1991 Oct. 1;174(4):915-24.
Go N F, Castle B E, Barrett R. Kastelein R, Dane W. Mosmann T R, Moore K W, Howard M. Interleukin 10, a novel B cell stimulatory factor: unresponsiveness of X chromosome-linked immunodeficiency B cells. J Exp Med 1990 Dec. 1;172(6):1625-31.
Mertz P M, DeWitt D L, Stetler-Stevenson W G, Wahl L M. Interleukin 10 suppression of monocyte prostagiandin H synthase-2 Mechanism of inhibition of prostaglandin-dependent matrix metalloproteinase production. J Biol Chem 1994 Aug. 19;269(33):21322-9.
Ma X, Aste-Amezaga M, Gri G, Gerosa F, Trinchieri G. Immunomodulatory functions and molecular regulation of IL-12. Chem Immunol 1997;68:1-22.
Schmitt E, Rude E, Germann T. The immunostimulatory function of IL-12 in T-helper cell development and its regulation by TGF-beta, IFN-gamma and ILA. Chem Immunol 1997;68:70-85.

D'Andrea A, Ma X, Aste-Amezaga M, Paganin C, Trinchieri G. Stimulatory and inhibitory effects of interleukin (IL)₄ and IL-13 on the production of cytokines by human peripheral blood mononuclear cells: priming for IL-12 and tumor necrosis factor alpha production. J Exp Med 1995 Feb. 1;181 (2):537-46.
Leonard J P, Waldburger K P, Schaub R G, Smith T, Hewson A K, Cuzner M L, Goldman S J. Regulation of the inflammatory response in animal models of multiple sclerosis by interleukin-12. Crit Rev Immunol 1997;17(5-6):545-53.
Donnelly R P, Fenton M J, Finbloom D S, Gerrard T L. Differential regulation of IL-1 production in human monocytes by IFTN-gamma and IL-4. J Immunol 1990 Jul. 15;145(2):569-75.
Wahl L, Corcoran M E, Mergenhagen S E, Finbloom D S. Inhibition of phospholipase activity in human monocytes by IFN-gamma blocks endogenous prostaglandin E2-dependent collagenase production. J Immunol 1990 May 1;144(9):3518-22.

Neale T J, Ruger B M, Macaulay H, Dunbar P R, Hasan Q Bourke A, Murray-McIntosh R P, Kitching A R. Tumor necrosis factor-alpha is expressed by glomerular visceral epithelial cells in human membranous nephropathy. Am J Pathol 1995 June; 146(6):1444-54;
Schall T J, Lewis M, Koller K J, Lee A, Rice G C, Wong G H, Gatanaga T, Granger G A, Lentz R, Raab H, et al. Molecular cloning and expression of a receptor for human tumor necrosis factor. Cell 1990 Apr. 20;61(2):361-70.
Wong G H. Goeddel D V. Tumour necrosis factors alpha and beta Inhibit virus replication and synergize with interferons. Nature 1986 Oct. 30-Nov. 5;323(6091):819-22.
Livingston D H, Appel S H, Sonnenfeld G, Malangoni M A. The effect of tumor necrosis factor-alpha and interferon-gamma on neutrophil function. J Surg Res 1989 April;46 (4):322-6.
Cicco N A, Lindemann A, Content J, Vandenbussche P, Lubbert M, Gauss J, Mertelsmann R, Herrmann F. Inducible production of interleukin-6 by human polymorphonuclear neutrophils: role of granulocyte-macrophage colony-stimulating factor and tumor necrosis factor-alpha. Blood 1990 May 15;75(10):2049-52.

Mangan D F, Welch G R, Wahl S M. Lipopolysaccharide, tumor necrosis factor-alpha, and IL-1 beta prevent programmed cell death (apoptosis) in human peripheral blood monocytes. J Immunol 1991 Mar. 1;146(5):1541-6.
Dinarello C A, Cannon J G, Wolff S M. New concepts on the pathogenesis of fever. Rev Infect Dis 1988 January-February;10(1):168-89.

1-21. (canceled)

22. An injectable formulation comprising a bacterial strain or an active derivative, fragment or mutant thereof.

23. The formulation as claimed in claim 22 wherein the strain is a probiotic bacterial strain.

24. The formulation as claimed in claim 22 wherein the strain is a lactobacillus strain.

25. The formulation as claimed in claim 22 wherein the strain is a Lactobacillus salivarius strain.

26. The formulation as claimed in claim 22 wherein the strain is Lactobacillus salivarius UCC118.

27. The formulation as claimed in claim 22 wherein the strain is in the form of bacterial cells.

28. The formulation as claimed in claim 27 wherein the cells are viable/live cells.

29. The formulation as claimed in claim 27 wherein the cells are non-viable/dead cells.

30. The formulation as claimed in claim 22 comprising a single strain.

31. The formulation as claimed in claim 22 comprising at least two different strains of the same or different species/genus or sub-genus.

32. The formulation as claimed in claim 22 comprising a prebiotic material.

33. The vaccine comprising a formulation as claimed in claim 22.

34. An injectable formulation of immunomodulatory bacteria.

35. The use of a formulation as claimed in claim 22 in the prevention and/or treatment of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris.

36. The use of a formulation as claimed in claim 22 in the prevention and/or treatment of disorders associated with intestinal inflammation.

37. The use of a formulation as claimed in claim 22 in the prevention and/or treatment of colitis.

38. The use of a formulation as claimed in claim 22 in the prevention and/or treatment of arthritis.

39. The method for the prophylaxis and/or treatment of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris comprising administering a formulation as claimed in claim 22.

40. The method for the prophylaxis and/or treatment of disorders associated with intestinal inflammation comprising administering a formulation as claimed in claim 22.

41. The method for the prophylaxis and/or treatment of colitis comprising administering a formulation as claimed in claim 22.

42. The method for the prophylaxis and/or treatment of arthritis comprising administering a formulation as claimed in claim 22.
